(19) United States
(12) Patent Application Publication
(10) Pub. No.: US 2020/0254232 A1
(43) Pub. Date: Aug. 13, 2020

(54) PERCUTANEOUS APPLIANCE WITH TRANSDERMAL COLLAPSIBLE FLANGES

(71) Applicant: CARDIAC ASSIST HOLDINGS, Ann Arbor, MI (US)

(72) Inventors: Barry N. Gellman, Ann Arbor, MI (US); Kurt A. Dasse, Ann Arbor, MI (US); Allen Kantrowitz, Ann Arbor, MI (US)

(73) Assignee: CARDIAC ASSIST HOLDINGS, Ann Arbor, MI (US)

(21) Appl. No.: 16/639,654

(22) PCT Filed: Aug. 16, 2018
(86) PCT No.: PCT/US18/00155

§ 371 Date: Feb. 17, 2020

Related U.S. Application Data
(60) Provisional application No. 62/546,168, filed on Aug. 16, 2017.

(51) Int. Cl. A61M 39/02; A61L 31/16; A61L 31/14

(52) U.S. Cl. CPC A61M 39/0247; A61L 31/16; A61M 2205/0266; A61M 2039/0261; A61L 31/146

(57) ABSTRACT

A percutaneous access device (PAD) is provided with a reduced likelihood of dislodgment that requires a reduced implantation incision so as to increase the rate of healing and reducing related infection. The PAD has a transdermal anchor in the form of flanged wings that are deployed following the implantation of the device in a patient. A deployment tool is provided to insert the PAD into the subject, and to actuate the flanges. The deployment tool protects the surface of the PAD with an outer cylindrical wall during insertion. The deployment tool has a stabilizer handle that is keyed with a key for insertion into the body or channel of the PAD. The stabilizer key stabilizes the PAD and prevents the PAD from rotating when the outer cylinder is rotated to turn an intermediate disc ring to actuate the flanges outward.

### CROSS-REFERENCE TO RELATED APPLICATION

This application claims priority of U.S. Provisional Patent Application Ser. No. 62/546,168 filed Aug. 16, 2017, which is incorporated herein by reference.

### FIELD OF THE INVENTION

The invention relates in general to percutaneous access devices (PAD), and in particular to devices with transdermal deployable anchor flanges for providing improved device stability with a limited incision for preventing infection at the site of percutaneous access.

### BACKGROUND OF THE INVENTION

A common problem associated with implantation of a percutaneous access device (PAD) is skin regeneration about the periphery of the device to form an immunoprotective seal against infection. New cell growth and maintenance is typically frustrated by the considerable mechanical forces exerted on the interfacial layer of cells. In order to facilitate skin regeneration about the exterior of a PAD, subject cells are often harvested and grown in culture onto PAD surfaces for several days prior to implantation in order to allow an interfacial cell layer to colonize PAD surfaces in advance of implantation. Unfortunately, cell culturing has met with limited acceptance owing to the need for a cell harvesting surgical procedure preceding the implantation procedure. Additionally, maintaining tissue culture integrity is also a complex and time-consuming task.

As an alternative to cell culturing on a percutaneous access device, vacuum assisted wound treatment about a percutaneous access device has been attempted. While DACRON® based random felt meshes have been used to promote cell regrowth in the vicinity of a wound, such felts have uncontrolled pore sizes that harbor bacterial growth pockets.

U.S. Pat. No. 7,704,225 to Kantrowitz solves many of these aforementioned problems by providing cell directing contours, porous biodegradable polymers and the application of vacuum to promote cellular growth towards the surface the neck of a PAD. The facilitating of rapid cellular colonization of a PAD neck allows the subject to act as their own cell culture facility, and as such affords more rapid stabilization of the PAD, and lower incidence of separation and infection.

An existing prior art percutaneous access device (PAD) is shown in FIG. 1 generally at 10. Device 10 has a central medical appliance 12 inserted that is depicted as a cannula shown in the context of providing fluid communication between a medical appliance 12 with subject vein V. The medical appliance 12 illustratively includes a catheter, cannula, pin, or wire or other percutaneous appliance with specific versions thereof including a Steinman pin and a Kirschner wire. The device 10 is percutaneous through the skin line (SL) and the epidermis, dermis, and subcutaneous layers that are denoted at E, D, and S, respectively. The device 10 is susceptible to lateral dislodgement (shown as bi-directional arrow 14) since there is no structure to stabilize the device 10 to the subject. Any lateral movement of the device 10 relative to the subject may tear tissue that has formed around the device 10 to seal the access point from infection and foreign bodies. Furthermore, healing times will be increased since the patient's movements will not allow the incision about the insertion device to seal as fast as a fully stationary device. FIG. 2 illustrates an existing prior art percutaneous device 20 with a transdermal base structure 22 that acts to stabilize the device 20 in a subject. However, the transdermal base structure 22 requires a larger incision in the patient for insertion of the device 20 as compared to device 10 of FIG. 1, as evidenced by dashed line 24. A larger incision increases healing time, as well as the chance for infection.

Thus, there exists a need for devices and process for stabilizing percutaneous devices to prevent or reduce the likelihood of infection related to percutaneous access devices while minimizing the required implantation incision.

### SUMMARY OF THE INVENTION

A percutaneous access device (PAD) is provided with a channel configured for the insertion of a medical appliance, and an articulating stabilizing anchor formed from a plurality of pivoting flanges joined to an intermediate ring via a flange pivot pin. A set of leading edges of the plurality of pivoting flanges are chamfered or have a sharpened edge adapted to force skin tissue apart when the plurality of pivoting flanges flare outward.

A deployment tool is provided for the percutaneous access device (PAD) described above. The deployment tool has an outer cylindrical wall dimensioned to fit over a sleeve that forms the channel of the PAD, a stabilizer handle that is keyed with a key for insertion in the channel, and a finger or a tool grasping position used to rotate the outer cylinder placed around the PAD. A set of protruding engagements or teeth extending from a bottom edge or perimeter of the outer cylinder are configured to engage a complementary set of grooves in the intermediate ring of the PAD to enable rotation and radial advancement of the flanges when the finger or the tool grasping position is rotated. The stabilizer handle prevents rotation of the PAD during deployment of the flanges.

### BRIEF DESCRIPTION OF THE DRAWINGS

The present invention is further detailed with respect to the following drawings that are intended to show certain aspects of the present invention, but should not be construed as a limit on the practice of the present invention.

FIG. 1 is a cross-sectional view of a prior art percutaneous access device with the relative dimensions of aspects exaggerated for visual clarity;

FIG. 2 is a cross-sectional view of a prior art percutaneous access device with relative dimensions of aspects exaggerated for visual clarity depicting a stabilizing transdermal base structure;

FIG. 3A is a perspective view of a percutaneous access device with expandable flanges in a closed position in accordance with embodiments of the invention;

FIG. 3B is a bottom view of the percutaneous access device of FIG. 3A with the expandable flanges in a closed position in accordance with embodiments of the invention;

FIG. 4A is a perspective view of the percutaneous access device of FIG. 3A with the expandable flanges in an open and deployed position in accordance with embodiments of the invention;

FIG. 4B is a bottom view of the percutaneous access device of FIG. 3A with the expandable flanges in an open and deployed position in accordance with embodiments of the invention;

FIG. 4C is a cross sectioned view of the percutaneous access device of FIG. 3A with the expandable flanges in an open and deployed position in accordance with embodiments of the invention;

FIG. 5 is a perspective view of a single deployable flange with an aperture in accordance with embodiments of the invention;

FIG. 6 is a perspective view of a percutaneous access device with expandable flanges deployed in a subject and sutured in place in accordance with embodiments of the invention;

FIG. 7 is an expanded perspective view of a deployment tool with an embodiment of the percutaneous access device with the expandable flanges in accordance with embodiments of the invention;

FIG. 8A is a cross sectioned view of the percutaneous access device with flexible flanges in an open and deployed position in a subject in accordance with embodiments of the invention;

FIG. 8B is a top view of the percutaneous access device of FIG. 8A with the flexible flanges deployed in a radial pattern in accordance with embodiments of the invention;

FIG. 9 is a cross sectioned view of a percutaneous access device with an oblique cylindrical sleeve with flexible flanges in an open and deployed position in a subject in accordance with embodiments of the invention;

FIG. 10 is a partial top view of a percutaneous access device with barbed flexible flanges in an open and deployed position in a subject in accordance with embodiments of the invention; and

FIGS. 11A and 11B are partial top views of the percutaneous access device of FIG. 10 with barbed flexible flanges partially retracted in steps to extend the barbs outward in the subject's subcutaneous tissue in an open and deployed position in a subject in accordance with embodiments of the invention.

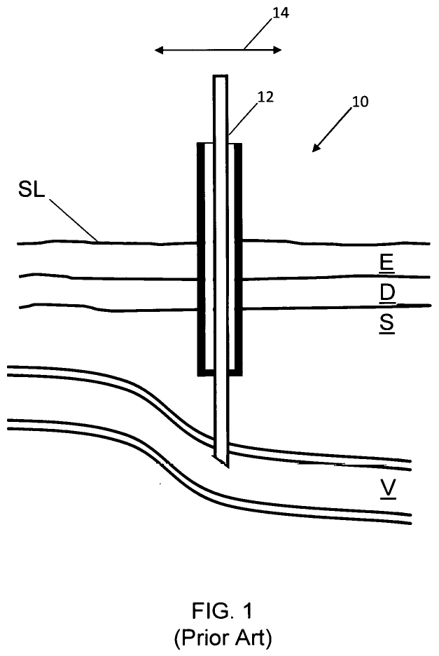

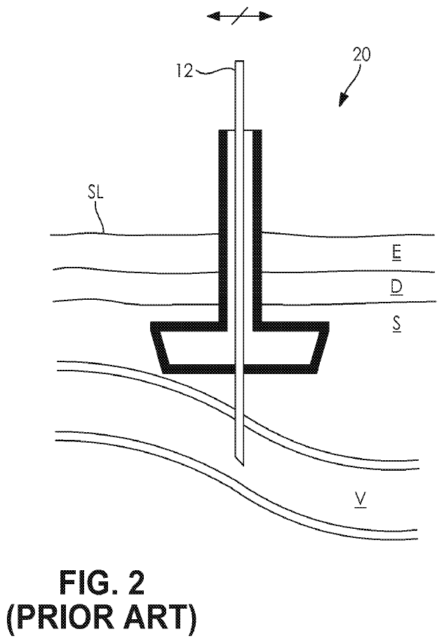

### DESCRIPTION OF THE INVENTION

The following description of the inventive embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. The inventive devices are disclosed herein in general with respect to a catheter, but this is not meant to be a limitation on the invention. Any tube, instrument, wire, material or assembly that penetrates the skin of a subject is similarly operable for use with the inventive device or integral therewith.

The invention has utility as a percutaneous access device with a reduced likelihood of dislodgment that require's a reduced implantation incision so as to increase the rate of healing and reducing related infection. Embodiments of the inventive percutaneous device have a transdermal anchor in the form of flanged wings that are deployed following the implantation of the device in a patient.

Embodiments of the inventive percutaneous access device are intended for use with a percutaneous instrument. Any appliance that is intended to traverse the skin is operable with the inventive device. The device is optionally used with a percutaneous access device illustratively a PICC, cannula, or other catheter, or pin illustratively a Steinman pin, Kirschner wires, and other devices or appliances that penetrate the skin. It is appreciated that the device is similarly operable with a bladder or other catheterization instrument. It is appreciated that catheterization instruments may include one or more catheters associated with more elaborate medical implants such as cardiac assist devices, peritoneal dialysis systems, hemodialysis systems, pulmonary assist devices (E.C.M.O.), hepatic assist devices, osseointegrated amputation prosthesis, CNS neural implant devices, peripheral neural implant devices, or catheters designed for other medical procedures.

The inventive devices decrease or prevent penetration or complications due to the presence of an agent. As used herein an “agent” is illustratively: an infectious agent such as bacteria, virus, fungus, other organism; or foreign material. Illustrative examples of foreign material include: bandage; soil; water, saliva, urine, or other fluid; feces; chemicals; or other matter known in the art. Illustrative examples of infectious agents that are prevented from penetrating or produce complications include P. aeruginosa, E. cloacae; E. faecalis; C. albicans; K. pneumonia; E. coli; S. aureus; or other infectious agents.

An inventive device is optionally used on the epidermis of a subject. As used herein, the term “subject” refers to a human or non-human animal, optionally a mammal including a human, non-primate such as cows, pigs, horses, goats, sheep, cats, dogs, avian species, and rodents; and a non-human primate such as monkeys, chimpanzees, and apes; and a human, also denoted specifically as a “human subject”.

As used herein, an “insertion site” is defined as an intentional interruption of skin or other tissue for the placement of a medical appliance.

An inventive device includes one or more sleeves. A sleeve is optionally an inner sleeve or an outer sleeve. As used herein, the terms “inner” and “outer” are relative terms in terms of encompassing relative dimensions and should not be construed contextually as to positioning relative to the epidermis. An inner sleeve is optionally made of a porous material or scaffold that is optionally penetrated by fluids or gasses. A scaffold is optionally a tissue scaffold that allows or promotes attachment of cells, illustratively, fibroblasts to the surface of an inner sleeve. An inner sleeve is optionally treated. An inner sleeve treatment illustratively includes compounds or surface textures that promote attachment of fibroblasts or other cellular material. Optionally, the inner sleeve is made of a woven material. A woven material is optionally penetratable by cells, fluids, gas, or other materials.

It is appreciated that an inner sleeve is optionally the only sleeve present in the device. An inner sleeve is optionally a porous scaffold that is suitable for moving fluid or gas through the sleeve away from the surrounding environment. Materials operable for use as an inner sleeve illustratively include: collagen, PEBAX, nylons, polypropylenes, polyurethanes, polyethylenes (HDPE, UHWPE, LDPE, or any blend of the aforementioned polyethylenes), PET, NiTi, MYLAR, Nickel Titanium Alloy, other polymers such as other thermoplastic polymers, fabrics, silicones such as silicone rubber, latex, glass, or other materials known in the art. It is appreciated that polymeric materials with a gradient of cross-linking density through the material afford certain advantages with respect to promoting vacuum or hydrodynamic draw and fibroblast infiltration. By way of example, a polymer having a greater rigidity proximal to the central axis of the device relative to the distal surface inhibits pressure differential induced collapse. In some inventive embodiments, an inner sleeve is made from chemically inert material. In some inventive embodiments, the porous scaffold is in direct contact with the skin of the subject or traverses the skin of the subject. In some inventive embodiments, an inner sleeve is textured or woven in such a way so as to provide attachment sites for fibroblasts. A texture is optionally a nanotexture. Illustrative nanotextures have pore sizes that are uniformly less than 500 nanometers to provide an anchor point for a fibroblast pseudopod extension, while having dimensions that disfavor bacterial colonization. A nanotextured surface as used herein has features indentations of from 50 to 500 nanometer median dimension. In some inventive embodiments, the indentations have a median dimension of between 100 and 300 nanometers.

In some inventive embodiments, a texture is in the form of a scaffold. A scaffold is illustratively formed of gold. A gold scaffold is optionally formed by making a sleeve from a gold/silver alloy that is dipped in an acid such as a mineral acid which selectively dissolves the silver leaving a gold structure with appropriate porosity. Alternatively, a scaffold is formed from an acid etchable, biocompatible nanocrystal such as silver or silica is dispersed in a polymer melt such as polycarbonate and a neck either formed directly therefrom, or the nanocrystal-doped polymer is coated onto a neck substrate. Through subjecting the nanocrystal-doped polymer to an acid or base solution, depending on the solubility of the nanocrystal, voids are formed in the polymer reflective of the original nanocrystal dopant. For instance, silver is readily dissolved in 6 N hydrochloric acid while silica is dissolved in concentrated hydrofluoric acid. Dissolution in the presence of sonication is appreciated to facilitate the process. Nanocrystal loading of 1 to 10 percent by weight, depending on the specific nanocrystal dimensions, is sufficient to achieve the desired uniformity and density of pores. Other porous surfaces and methods of manufacture are illustrated in U.S. Pat. No. 7,704,225 and references cited therein, each of which are incorporated herein by reference in their entirety.

It is appreciated that an inner sleeve is optionally coated or impregnated with a first compound. Coating or impregnating optionally provides lubrication so as to ease insertion of the appliance into the skin. A compound optionally: is antibacterial such as those described in WO 2008/060380, the contents of which are incorporated herein by reference; resist or promote cellular adhesion; are anticoagulants or procoagulants; or other desirable compounds.

A compound optionally includes factors operable to selectively promote fibroblast growth and/or decrease attachment of bacteria or other contaminants. A compound optionally promotes growth of cells such as fibroblasts. A coating optionally includes the compound fibroblast growth factor (FBF). Optionally, FBF is used in a coating along with insulin and/or dexamethasone. The presence of dexamethasone and/or insulin will promote multiple layer growth of fibroblasts on the surface of or within the pores of a sleeve.

Coating substances illustratively include cell growth scaffolding matrices as detailed in U.S. Pat. Nos. 5,874,500; 6,056,970; and 6,656,496; and Norman et al. Tissue Eng. 3/2005, 11(3-4) pp. 375-386, each of which is incorporated herein by reference. An exemplary coating is a tissue scaffolding, poly-p xylylene, parylene and chemical modified versions of such coatings to enhance post-insertion stabilization. Chemical modifications illustratively include bonding of fibronectin and other molecules implicated in the healing process. While tissue scaffolding and polymers are readily applied by painting, dip coating, and spraying, it is also appreciated that polymeric coating are also readily applied by gas phase deposition techniques such as chemical vapor deposition (CVD). A coating is optionally porous in order to enhance capillary draw. In some inventive embodiments, a coating is biodegradable. A coating optionally has pores typically of an average size of between 10 and 500 microns, optionally, of an average size of between 30 and 50 microns.

An inventive device optionally includes an outer sleeve. An outer sleeve functions to segregate or deliver vacuum draw pressure to an inner sleeve. The outer sleeve optionally circumferentially and longitudinally covers an inner sleeve. This configuration optionally shields the inner sleeve from epidermal bacterial or other agents upon insertion.

An outer sleeve is optionally tapered at one or both ends. Tapering at a distal end (the end nearest the internal end of the catheter during use) provides improved insertion of the appliance into the skin of a subject. A taper may form a smooth interaction with the catheter at the outer sleeve distal end or a ridge is optionally present at or near the site of device interaction with the catheter.

An outer sleeve is optionally made of any material suitable for use with a percutaneous appliance. Illustrative materials operable for an outer sleeve include such materials that have a memory or are self-expanding. Materials operable for use as an outer sleeve illustratively include: PEBAX, nylons, polyurethanes, polyethylenes (HDPE, UHWPE, LDPE, or any blend of the aforementioned polyethylenes), PET, NiTi, MYLAR, Nickel Titanium Alloy, other polymers such as other thermoplastic polymers, fabrics, silicones such as silicone rubber, latex, glass, or other materials known in the art. An outer sleeve optionally includes or is formed of a scaffold. An outer sleeve scaffold is optionally made of the same or different material as an inner sleeve scaffold. Scaffolds operable for an inner sleeve are similarly operable for an outer sleeve.

It is appreciated that an outer sleeve is optionally coated or impregnated with a second compound. A second compound is optionally the same as a first compound. Coating or impregnation optionally provides lubrication so as to ease insertion of the appliance into the skin. A compound optionally: is an antibacterial coating or impregnated material such as those described in WO 2008/060380, the contents of which are incorporated herein by reference compounds to resist or promote cellular adhesion; anticoagulants or procoagulants; or other desirable compound.

In some inventive embodiments, an outer sleeve is textured. A texture is optionally formed of a tissue scaffold. A texture on an outer or inner sleeve optionally has pore sizes, ridges, depressions, indentations, or other texture that is uniform or non-uniform. A texture is optionally of a depth less than 500 nanometers to provide an anchor point for a fibroblast pseudopod extension, while having dimensions that disfavor bacterial colonization. A nanotextured surface as used herein has a uniform distribution of 50 to 500 nanometer median dimension indentations. In some inventive embodiments, the indentations have a median dimension of between 100 and 300 nanometers.

In some inventive embodiments, an outer sleeve surrounds an inner sleeve. The outer sleeve and inner sleeve are optionally formed from a unitary piece of material. The outer sleeve is optionally oriented surrounding an inner sleeve and optionally is slidably positionable about an inner sleeve. In some inventive embodiments, an outer sleeve protects an inner sleeve upon insertion of the inventive appliance and is positionally adjusted relative to the inner sleeve illustratively to a mark or other region that is optionally positioned above the epidermis. In some inventive embodiments, the inner sleeve remains traversing the skin while the outer sleeve is positioned above the epidermis or penetrates to one or more desired depths or levels.

An inventive device is optionally manufactured as a separate assembly or unitary piece so as to be associatable with a catheter prior to placement across the skin. An inventive device is optionally formed with a slot to accept a catheter or other appliance. An appliance is optionally slidable onto a catheter prior to inserting the catheter through the skin. Optionally, a catheter serves as a guide for an inventive instrument such that the appliance is slid onto a catheter following catheterization into the same insertion location. Engagement of the appliance prevents agents from entering the insertion point or will remove agents already in or under the insertion point.

Without intending to be bound to a particular theory, a surface of an inventive device in contact with compromised skin for device insertion promotes intercalation of fibroblasts regardless of whether the surface is textured, coated, or a combination thereof so as to simultaneously promote orthological changes in the fibroblast from circulatory form to dendritic and/or stellate forms through a depth of more than one layer of fibroblast at a time and preferably more than five layers of fibroblasts simultaneously anchoring to the device and more preferably more than ten such layers of fibroblasts. Fibroblast orthological changes simultaneously in more than one layer of such cells serve to rapidly stabilize the percutaneous inventive device. In conjunction with the vacuum pressure draw during the process, infection risks are minimized and an inventive device is stabilized against pullout or other device motions relative to the surrounding dermal layers.

An inventive device is optionally connected to a vacuum source. A vacuum source can be any source operable for creating negative pressure in or around the device. A vacuum source is optionally a passive vacuum such as a vacuum tube or bottle, or an active vacuum source illustratively a mechanical pump, a syringe, or other vacuum source. A vacuum source optionally applies a continuous or intermittent negative pressure. The magnitude of the negative pressure is optionally adjustable, constant, or variable. In some inventive embodiments, an intermittent vacuum is used. Alternatively, a hydrodynamic draw agent is provided that draws fluid from the tissue surrounding through the sleeve via the conduit. A hydrodynamic draw source illustratively includes a super absorbent polymer such as sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile; polycarbonate or polycarbonate/ABS blend; high osmotic pressure compositions, such as water soluble salts; and capillary flow draw agents such as dry silica, or other dry hydrophilic powders such cellulosic material.

In some inventive embodiments, several device diameters are operable. An inventive device optionally has an inner diameter and an outer diameter. The inner diameter of the device is optionally associated with the diameter of the percutaneous instrument. A larger percutaneous appliance generally will require a larger inner diameter of the device. Alternatively, a single inner diameter device is produced with removable and replaceable gaskets or seals that allow a wide range of catheter diameters or shapes to be used with the device.

In some inventive embodiments, the appliance is reusable. Embodiments of the inventive device are optionally autoclavable or otherwise sterilizable.

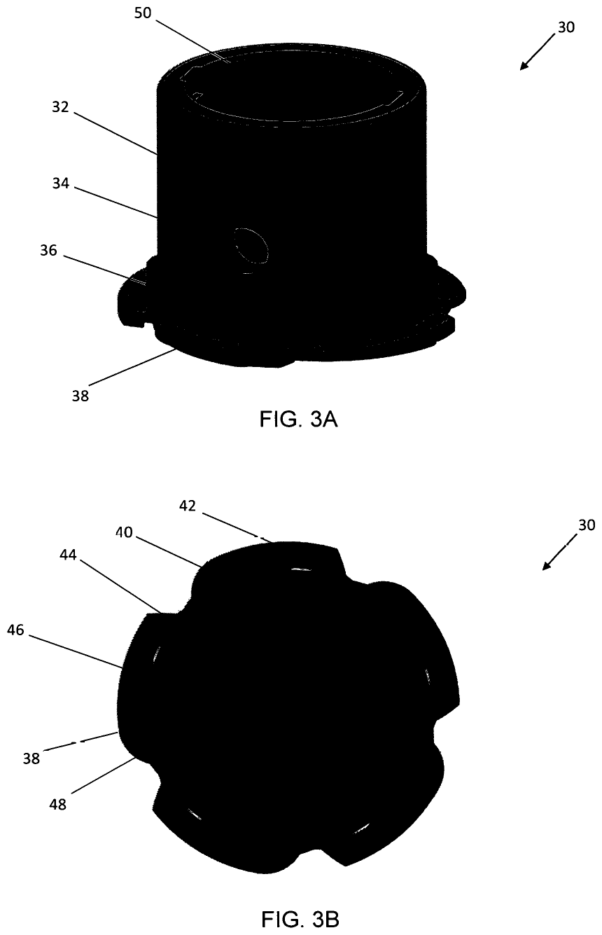

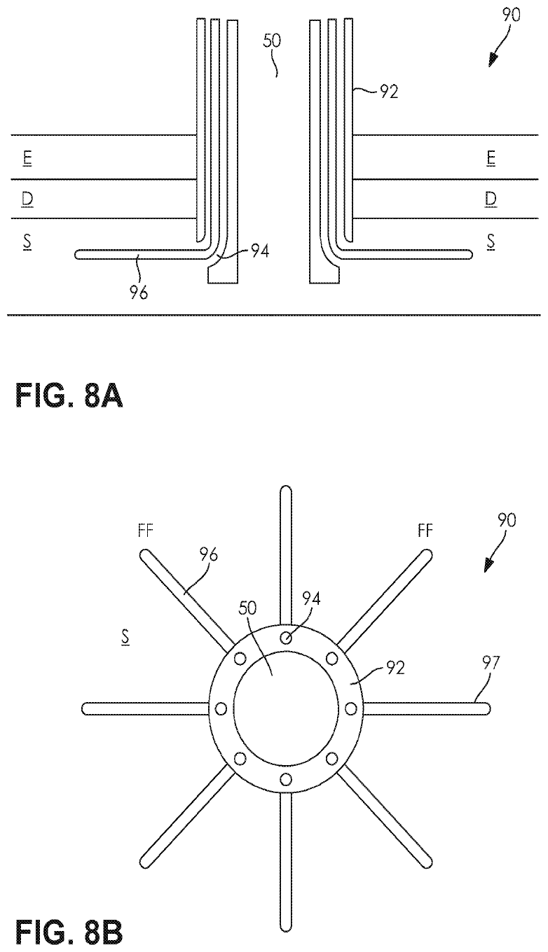

Referring now to the figures, FIGS. 3A and 3B show an embodiment of the inventive percutaneous access device 30 with an articulating transdermal stabilizing anchor made up of flanges 38 in a closed non-deployed position. A cylindrical sleeve 32 forms a channel 50 for insertion of a medical appliance. Medical appliances illustratively include a catheter, cannula, pin, wire, or other percutaneous appliance with specific versions thereof including a Steinman pin and a Kirschner wire. Port 34 provides an attachment point for a tube 78 (see FIG. 7) for electrical or pneumatic connections. The device 30 is percutaneous through the epidermis, dermis, and subcutaneous layers of a subject's skin when implanted. The articulating transdermal stabilizing anchor of flanges 38 are each joined to the underside of an intermediate ring 36 with a flange pivot pin 48, where the intermediate ring 36 is at the distal end of the sleeve 32. The intermediate ring 36 is free to rotate relative to the sleeve 32 and is held in place by a lower base 33 fixedly joined to the sleeve 32. In a specific inventive embodiment, the lower base 33 is ultrasonically joined to the sleeve 32. In a specific inventive embodiment, the sleeve 32 is fastened to the lower base 33 with a screw like connection to permit removal of the sleeve 32 after implantation for replacement (if needed), or for assembly in two pieces during the implant procedure. The sleeve 32 may take different forms based on the application. Adjacent apexes of the flanges 38 are joined by radial shaped flange connectors 40 with pivot pins 42, where each of the flange connectors 40 have a slot 46 in which guidance pins 44 from the flanges 38 travel. As shown in FIGS. 3A and 3B, the flanges 38 that provide the anchor are not deployed so as to minimize the incision required for the implantation of the device 30 in a subject.

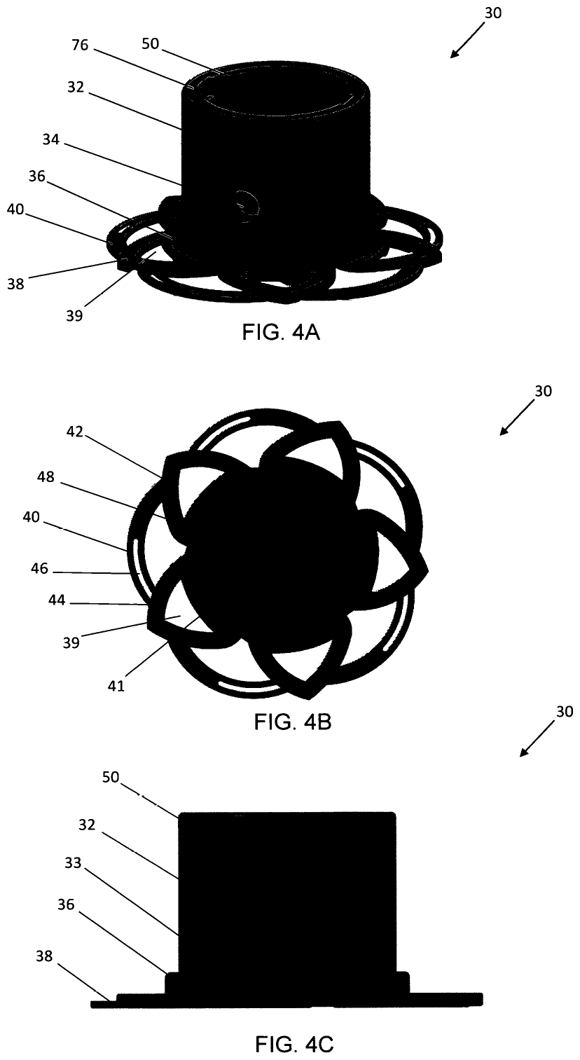

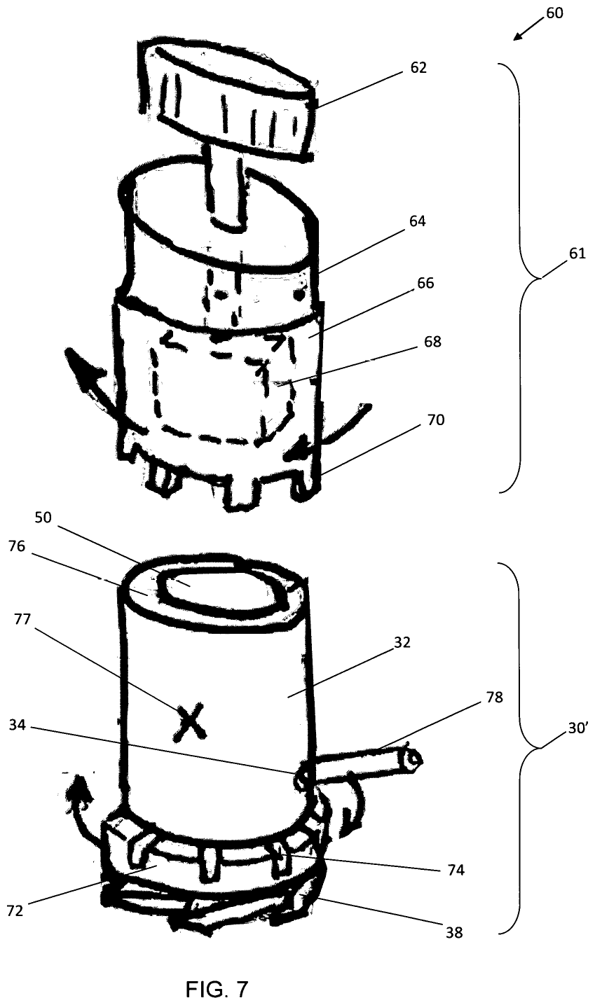

Following the implantation of the device 30 through the epidermis, dermis, and subcutaneous layers, the articulating transdermal stabilizing anchor flanges 38 are deployed outward as shown in FIGS. 4A-4C and FIG. 6. In the specific inventive embodiment, shown, rotation of the intermediate ring 36 is in a clockwise direction relative to the sleeve 32 opens or flares the flanges 38 outward. However, it is appreciated that in another embodiment a counter clockwise rotation of an embodiment of the intermediate ring may actuate the flanges outward. The rotation of the intermediate ring 36 causes the flanges 38 to pivot about the flange pivot pins 48 protruding from the intermediate ring 36, as a protrusion on the upper surface (not visible) of the flanges 38 moves along the spiral tracks 41 embedded in the lower base 33 to guide the flanges 38.

During the deployment phase the flanges 38 and flange connectors 40 articulate outward and dissect tissue below the skin line that may include the dermis and subcutaneous layers above the fascia (F). The leading edges of the flanges 38 and the flange connectors 40, which may be chamfered or have a sharpened edge, force the skin tissue apart. It is appreciated that the number of flange 38 wings or peddles may vary, with two or more opposing sets of flanges providing the most stabilizing support to counteract accidental lateral dislocation of the percutaneous access devices.

FIG. 6 illustrates a deployed embodiment of the inventive percutaneous access device (PAD) 30 under the skin line (SL) of a subject. Placement of the PAD 30 may be from outside to inside the subject, or from inside to outside where the device is placed under the skin away from the final implementation site and tunneled to the final location. In a specific inventive embodiment, it is recommended to create an incision hole in the skin that is 65% of the diameter of the PAD 30.

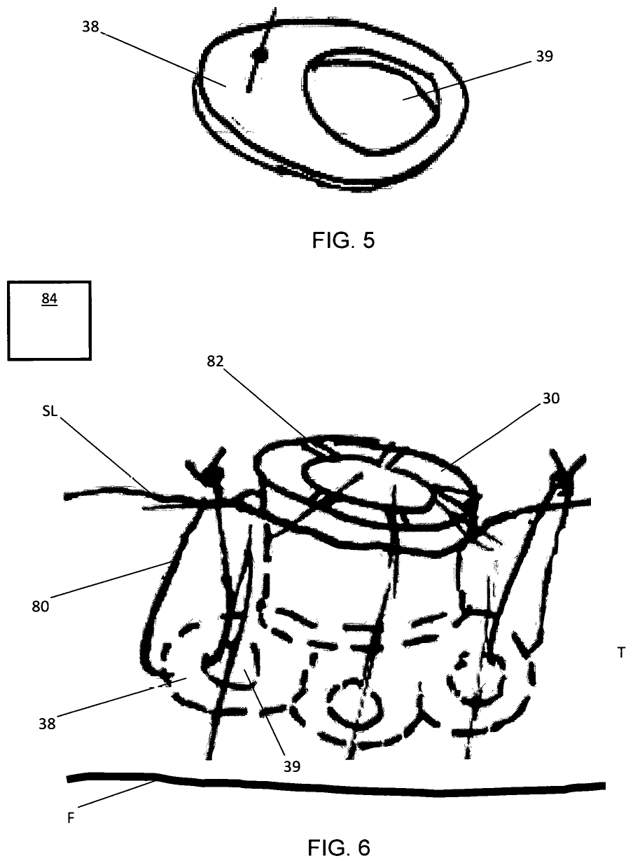

In order to encourage tissue ingrowth into the deployed flanges 38 and to stabilize and secure the PAD 30, tissue to tissue contact is provided through the apertures 39 in the flanges 38 as best seen in FIG. 5. In still other inventive embodiment, a mesh material (not shown for visual clarity) extends across at least a portion of the aperture 39, while in other embodiments the whole of aperture 39 is so covered. The mesh material affords both additional mechanical stabilization area, as well as additional fibroblast adhesion surface area. The thickness of the flanges 38 should be minimized so that tissue approximation may be achieved.

Without intending to be bound to a particular theory, the inclusion of through holes in a flange promote tissue-to-tissue healing that is not reliant on tissue adherence to the flange to provide stabilization. Additionally, it is believed that through holes also promote lymphatic drainage proximal to the insertion wound. It is appreciated that tissue-to-tissue approximation is further enhanced with the use of vacuum applied about the inventive device, thereby drawing the wound surfaces together.

In a specific implementation as shown in FIG. 6, temporary monofilament sutures 80, which are later removed or that dissolve, may be used to secure the PAD 30 during tissue ingrowth and healing. As shown the sutures 80 may be threaded through the apertures 39 of the flanges 38. Markers 82 on the top of the PAD 30 may be used to identify the under skin deployed positions of the apertures 39. In specific inventive embodiments, alternative anchoring devices may be used illustratively including staples or compression clamps to gain tissue to tissue contact.

With the deployment of the articulating anchor flanges 38, the possibility of accidental lateral dislocation of the percutaneous access device 30 is largely eliminated. The sleeve 32 and articulating anchor 38 may be formed of materials as detailed above with respect to the inner sleeve and are characterized by a porous matrix (shown as 76 in FIG. 7) adjacent to a perforated outer surface (shown as 77 in FIG. 7). In operation, the porous matrix experiences strong vacuum or hydrodynamic forces created by a vacuum or hydrodynamic draw source, collectively depicted at 84 in FIG. 6. The matrix has a rigidity sufficient to prevent collapse under the draw pressure as an operative requirement and thereby maintain vacuum or hydrodynamic through the matrix. A highly cross-linked polymeric substance, collagen, porous ceramic or metallic substances are particularly well suited to form a matrix. A matrix is appreciated to promote vacuum draw, such large pores are sized such that both bacteria and fibroblasts readily infiltrate such pores. Without intending to be bound by a particular theory, it is believed that deleterious agents within the matrix are actively drawn into the channel 50 or if capable of biological multiplication, inhibited from doing so by the forces exerted thereon by the source 84. The surface of the flanges 38 may have an optional nanotexture, as detailed above to promote fibroblast pseudopod extension adherence yet are sufficiently small to discourage bacterial colonization. It is appreciated that the surface of the flanges 38 experiences limited draw from the source 84. It is appreciated that the application of draw forces via source 84 in addition to inhibiting agents and promoting fibroblast infiltration also serves the tissue-scale function of adhering the body tissue T surrounding the device 30 in a fixed position in contact with device 30 so as to form a stable interface therebetween. The resulting interface is superior for the granulation process relative to conventional sutures or adhesive bandages. It is appreciated that the matrix is integral, or alternatively is formed as contiguous separate layer as detailed above with respect to sleeve 32. A coating substance optionally overcoats the outer surface of the sleeve 32, with coating substances illustratively include cellular ingress scaffolding, as will be further detailed below. The coating substance has a tissue contacting surface that is optionally nanotextured. The sleeve and the flanges may further may include coating compound that may be applied to the sleeve and flanges, where the compound is a growth factor, extracellular matrix factors, fibroblast receptors, fibronectin, laminectin, RGD factor, dexamethasone, or combinations thereof.

FIG. 7 is an expanded perspective view of a system 60 with a deployment tool 61 with an embodiment of the percutaneous access device 30′ with the expandable flanges 38. The deployment tool 61 may be used to insert the PAD 30′ into the subject, and to rotate the intermediate ring 72 to actuate the flanges 38. The deployment tool 61 protects the surface of the PAD 30′ with an outer cylindrical wall 66 during insertion. In a specific inventive embodiment, the outer cylindrical wall 66 is made with thin wall stainless steel that fits marginally over the PAD 30′. The deployment tool 61 has a stabilizer handle 62 that is keyed with key 68 for insertion into the body or channel 50 of the PAD 30′. The stabilizer key 68 stabilizes the PAD 30′ and prevents the PAD 30′ from rotating when the outer cylinder 66 is rotated to turn the intermediate disc ring 72 to actuate the flanges 38 outward. The finger or tool grasping position 64 is used to rotate the outer cylinder 66 placed around the PAD 30′. The cylinder body 66 has a set of protruding engagements or teeth 70 extending from the bottom edge or perimeter of the cylinder 66. The engagements or teeth 70 engage complementary grooves 74 in the intermediate disc ring 72 to enable rotation and radial advancement of the flanges 38. Both the key 68 and cylindrical wall 66 of the insertion tool 61 are removed once the PAD 30′ is secured in the subject.

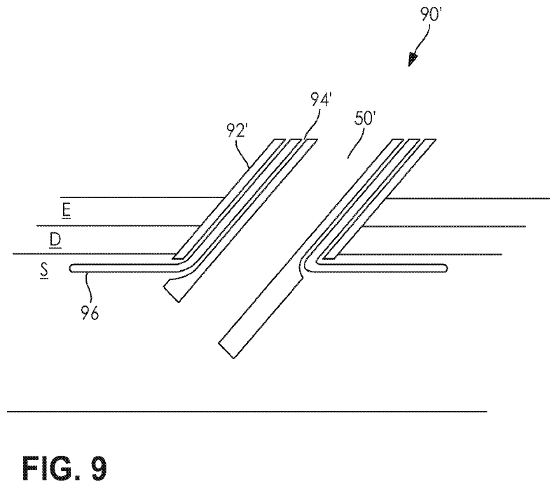

FIG. 8A is a cross sectioned view of the percutaneous access device 90 with flexible flanges 96 in an open and deployed position in a subject's skin. One or more flexible flanges 96 project radially outward from a corresponding conduit 94 in the sleeve 92 and into the subject's subcutaneous S tissue. In a specific inventive embodiment, once the percutaneous access device 90 is inserted in the subject, the one or more flexible flanges 96 are introduced into the conduits 94 on a portion of the PAD 90 that remains external to the epidermis E, and the flexible flanges 96 are then each advanced radially away from the PAD 90 and deployed into the subject's subcutaneous S tissue. FIG. 8B is a top view of the percutaneous access device 90 with the flexible flanges deployed 96 in a radial pattern in the subject's subcutaneous S tissue.

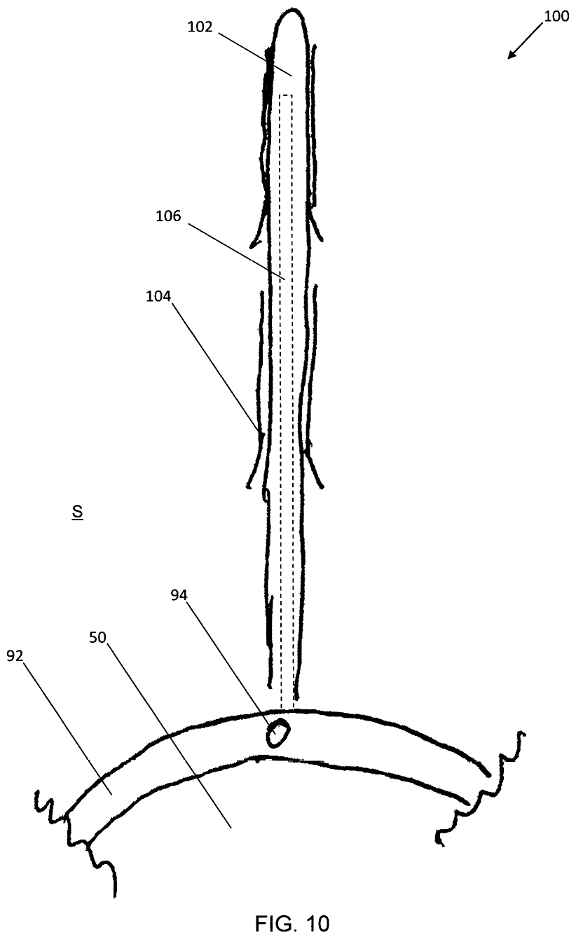

FIG. 9 is a cross sectioned view of a percutaneous access device 90′ with an oblique cylindrical sleeve 92′ with flexible flanges 96 in an open and deployed position in a subject's subcutaneous S tissue. In this inventive embodiment, the flexible flange 96 elements remain parallel to the plane of the dermis while the central axis of the main lumen or channel 50′ of the PAD 90′ is oblique to the plane of the dermis.

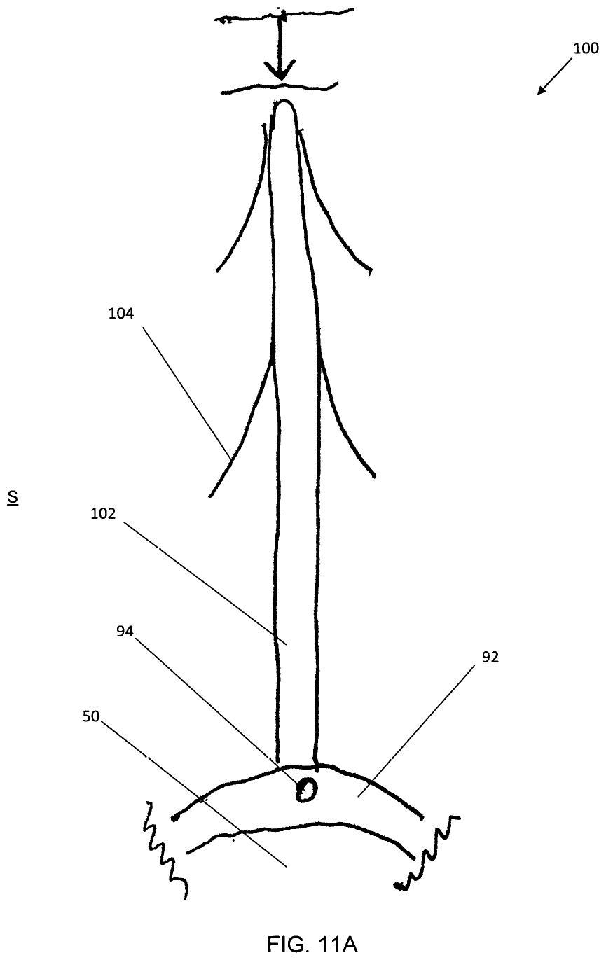

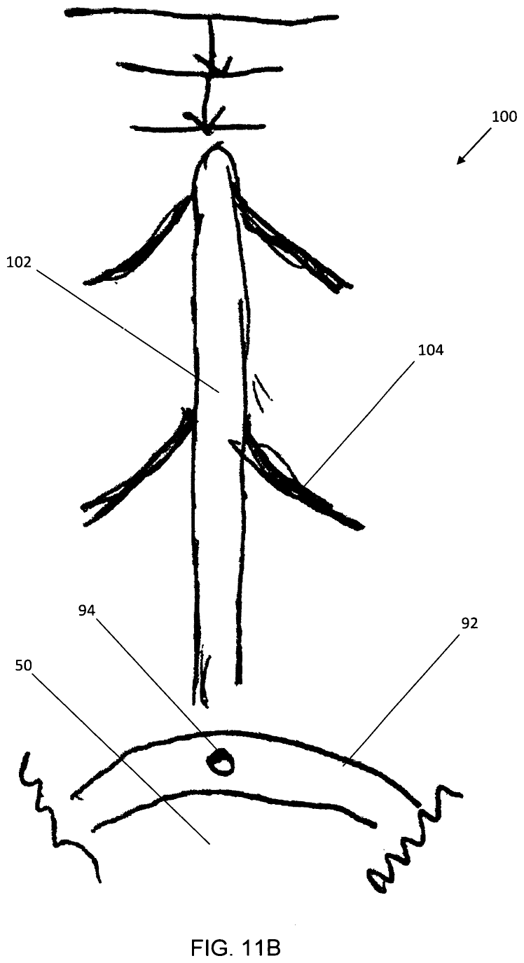

FIG. 10 is a partial top view of a percutaneous access device 100 with flexible flanges 102 with barbs 104 in a deployed position in a subject's subcutaneous S tissue. The barbs 104 are initially flush with the flexible flanges 102 when inserted into the PAD 100. As shown in FIGS. 11A and 11B the flexible flanges 102 are partially retracted to extend the barbs 104 outward in the subject's subcutaneous tissue to provide additional tissue anchorage. FIGS. 11A and 11B illustrate the extension of the barbs 104 as a two-step retraction, however it is appreciated that one continuous retraction of the flexible flange 102 may be used to fully deploy the barbs 104 in the subject's subcutaneous tissue.

Embodiments of the inventive flexible flange (96, 102) may be made of non-biodegradable materials known to permit investiture with fibroblasts and collagen deposition. The sleeve and the flexible flanges may have a coating compound that is a growth factor, extracellular matrix factors, fibroblast receptors, fibronectin, laminectin, RGD factor, dexamethasone, or combinations thereof.

Each of the flexible flange elements (96, 102) may have a central lumen which can accommodate a shape-memory-alloy stiffener 106 (shown in FIG. 10) which serves, during implantation, to help direct the flange element into the subcutaneous tissue. Optionally, the shape-memory-alloy stiffener 106 can be withdrawn after implantation use. It is appreciated that the central lumen of the flange element (96, 102) can serve further to conduct vacuum from a draw source 84 as shown in FIG. 6 into the flexible flange element (96, 102) to optimize fibroblast collagen production and fibrous integration with the flexible flange element.

In specific inventive embodiment, the tip of the flange element 96 may have an extension 97 as shown in FIG. 8B, biodegradable or detachable, which serves to hold the flange element fully extended during fibroblast integration with the subcutaneous tissue.

In specific inventive embodiments, the conduits 94 guiding the flexible flange elements (96, 102) down the wall of the PAD may be one or more non-intersecting helical pathways which guide the flexible flange elements to their exit portals of the PAD 90 at the level of the subcutaneous tissue. It is appreciated that the flexible flange elements may exit the subcutaneous rim at an angle other than ninety degrees to the long axis of the main medical appliance lumen of the PAD.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A percutaneous access device (PAD) comprising:
a channel configured for the insertion of a medical appliance; and
an articulating stabilizing anchor formed from a plurality of pivoting flanges joined to an intermediate ring via a flange pivot pin.

2. The device of claim 1 wherein a set of leading edges of said plurality of pivoting flanges are chamfered or have a sharpened edge adapted to force skin tissue apart when said plurality of pivoting flanges flare outward.

3. The device of claim 1 wherein said plurality of pivoting flanges further comprise an aperture therein.

4. The device of claim 1 further comprising a lower base with a series of spiral tracks embedded on said lower base.

5. The device of claim 1 wherein said lower base is fixedly attached to a sleeve with a screw connection or with an ultrasonic bond.

6. (canceled)

7. The device of claim 1 wherein said PAD further comprises a sleeve that forms said channel, an intermediate ring positioned at a distal end of the sleeve that forms said channel, and a set of markers for suture placement.

8. The device of claim 1 wherein said plurality of pivoting flanges are treated or shaped to encourage fibroblast attachment.

9. (canceled)

10. The device of claim 1 wherein said plurality of pivoting flanges further comprises a coating substance.

11. The device of claim 10 further comprising a coating compound; and
wherein said compound is at least one of growth factor, extracellular matrix factors, fibroblast receptors, fibronectin, laminectin, RGD factor, dexamethasone, or combinations thereof.

12. The device of claim 1 wherein said plurality of pivoting flanges flare outward when said intermediate ring is rotated.

13. The device of claim 1 wherein said intermediate ring rotates relative to a sleeve and is held to said sleeve by a lower base fixedly attached to said sleeve.

14. A deployment tool for the percutaneous access device (PAD) device of claim 1 comprising:
an outer cylindrical wall dimensioned to fit over said sleeve;
a stabilizer handle that is keyed with a key for insertion in said channel;
a finger or a tool grasping position used to rotate the outer cylinder placed around said PAD; and
a set of protruding engagements or teeth extending from a bottom edge or perimeter of said outer cylinder that engage a complementary set of grooves in said intermediate ring to enable rotation and radial advancement of said flanges when said finger or said tool grasping position is rotated, and where said stabilizer handle prevents rotation of said PAD.

15. (canceled)

16. A percutaneous access device (PAD) comprising:
a channel configured for the insertion of a medical appliance;
one or more conduits in said channel extending from an upper portion of said PAD to a lower portion of said PAD; and
one or more flanges adapted for insertion in said one or more conduits.

17. The device of claim 16 wherein the one or more flanges deploy in a radial pattern.

18. The device of claim 16 wherein the one or more flanges further comprise a series of barbs on an outer surface of the flanges, said barbs adapted to expand outward into the subject's tissue when the flexible flange is partially retracted following insertion.

19. The device of claim 16 wherein the one or more flanges are made of non-biodegradable materials that permit investiture with fibroblasts and collagen deposition.

20. The device of claim 16 wherein the one or more flanges are flexible so as to deform and return to an original shape during deployment.

21. (canceled)

22. The device of claim 16 wherein the one or more flanges further comprises a central lumen adapted for a shape-memory-alloy stiffener.

23. The device of claim 22 wherein said central lumen conducts a vacuum from a draw source into said flexible flange element to optimize fibroblast collagen production and fibrous integration with said flexible flange element.

24. The device of claim 16 wherein the one or more flanges have through holes that permit tissue-to-tissue healing.

25. (canceled)
